(19) United States
(12) Patent Application Publication
(10) Pub. No.: US 2004/0063163 A1
(43) Pub. Date: Apr. 1, 2004

(54) Method for magnetising chemical or biological markers

(76) Inventors: Frederic Buffiere, Pessac (FR); Christine Betremieux, Bouvines (FR); Laetitia Gaillard, Canejan (FR); Gerard Ovlaque, Bondues (FR); Christophe Vinzia, Ennevelin (FR)

Correspondence Address: Blakely Sokoloff Taylor & Zafman, Seventh Floor, 12400 Wilshire Boulevard, Los Angeles, CA, 90025, US

(21) Appl. No.: 10/433,852

(22) PCT Filed: Dec. 7, 2001
(86) PCT No.: PCT/FR01/03887

(30) Foreign Application Priority Data
Dec. 8, 2000 (FR) 00 16026

(51) Int. Cl. G01N 33/567

(52) U.S. Cl. 435/7.21

(57) ABSTRACT

The invention concerns a method for magnetising chemical or biological markers using magnetic particles, said method comprising steps which consist in: activating the magnetic particles so as to modify their surface state; contacting the activated magnetic particles with the markers so as to produce non-specific bonds between them. The invention also concerns the use of biological markers magnetised by said method as reagents or analytes in a biological assay.

The invention relates to a process for magnetisation of chemical or biological markers and use of the said biological markers in a biological analysis test.

It relates particularly to cell magnetisation, particularly red blood cells carrying blood group antigens on their surface, or magnetisation of antibodies.

Immunology analysis tests then typically concern the searching for antigens on the surface of red blood cells in the blood or in a blood component, using magnetised antierythrocytary antibodies (for example, erythrocytary typing), or the search for anti-erythrocytary antibodies using magnetised red blood cells on which specific antigens are present and/or fixed.

Marker magnetisation processes based on magnetic particles in which the particles are related to markers through specific or covalent links, are already known.

This type of process uses magnetic particles for which the surface has been functionalised so as to form links with a specific marker.

Therefore, it has the disadvantage that it requires the preparation of specific magnetic particles as a function of the marker to be magnetised.

Furthermore, this specific or covalent coupling is sometimes difficult to make and depends on the use of a limited number of combinations (antigen-antibody pair, avidinbiotine pair, etc.). Therefore, this technique requires the possession. of a certain type of antibody or antigen, or that the biotin should specifically mark effector molecules.

Furthermore, conservation (conservation time, storage temperature, etc.) of these functionalised particles is largely dependent on the fragility of the molecule fixed to the particle surface.

Therefore, the invention is designed to overcome all these disadvantages, particularly by proposing a process for direct magnetisation of particulate and/or figured elements without the use of molecules that for example recognise antigen and/or antibody type structures, without masking and/or modifying the structures to be used for example during a biological analysis test.

Consequently, according to a first aspect, the invention proposes a process for magnetisation of chemical or biological markers using magnetic particles, the said process comprising steps consisting of:

activating magnetic particles so as to modify their surface condition;

putting activated magnetic particles into contact with markers so as to create non-specific links between them.

According to a second aspect, the invention proposes the use of biological markers magnetised by the use of such a process, such as reagents or analytes in a biological analysis test.

Other objectives and advantages of the invention will become clear from the following description.

The process is a means of magnetising markers using magnetic particles by the creation of non-specific links between the particles.

The markers may be chemical, for example in molecular form, or biological, for example in cellular form, and the magnetic particles may for example be compatible polymer beads loaded with a magnetic material.

A marker is in contact with its immediate environment through its surface, and in the case of a cellular element, through its membrane.

The membrane may be described as being a dual layer of phospholipids, above which floats a fluid mosaic of more or less glycosilised proteins. The molecular diversity of the membrane and its high fluidity depending on the environmental conditions, enable it to set up a large number of relations with the outside environment. The cell has a sort of arsenal of possible links with various surrounding molecular structures, such as magnetic particles.

Thus, the cell is capable of creating links with elements, including:

specific elements, for example when a receptor structure of the membrane is recognised by an effector specific to it (for example, in the case of hormone-receptor links or antigen-antibody links);

non-specific elements, by passive adsorption of the said molecules at its surface; for example, in the case of antigens in the Lewis system, adsorbed on the surface of human red blood cells.

This cellular membrane, which is the first meeting structure of the cell, will therefore be the source of important interactions both for the cell and for the external environment. Most molecular cell identification structures are on its surface, also with other structures capable of specifically or non-specifically bonding to foreign molecules.

In one particular example, markers are red blood cells for which the cellular membrane is the support of antigenic structures defining the various blood groups that are required before any blood transfusion.

At the present time, very many diagnostic systems to search for antibodies in a biological medium or antigen structures causing a host response, are based on the use of functionalised magnetic particles.

For example, for sorting a population of cells with the CD4 antigen in a human blood sample, it is possible to specifically fix a ferromagnetic particle on the surface of positive CD4 cells through an appropriate antibody. It is then sufficient to submit the entire blood sample to a magnetic field in order to isolate the population of cells that interacted with the particles.

These techniques are based exclusively on the use of a variety of different types of magnetic particles with a fundamental characteristic, namely ‘functionalisation’ of their contact surface by a recognition molecule.

In other words by a covalent fixation of molecules with recognition properties specific to the marker.

For example, antibody type proteins can be fixed to the surface of these particles which will be able to specifically recognise the antigens corresponding to them, thus enabling indirect fixation to the antigen structure (either particulate or soluble) through the magnetic particle.

Similarly, attachment of the avidin or streptavidin type molecule is a means of creating a specific bridge between the particles and any figured or soluble element with biotin molecules on its surface.

On the other hand, the process according to the invention uses large areas of phospholipids and/or proteins present on the surface of a red blood cell, the said zones not acting directly in the definition of blood groups and phenotypes.

These zones are very interesting if they are functionalised through links with external elements, thus conferring dynamic properties on the red blood cell, for example displacement properties in a particular environment.

If paramagnetic particles are non-specifically linked to these zones, the red blood cells thus magnetised have two properties, firstly they are attracted under the effect of a magnetic field, and that they carry the antigenic structures mentioned above on their surface.

Thus, the property of cellular membranes is used to be able to establish a large number of so-called non-specific interactions with particles so as to use the kinematic property of particles and thus transmit this ease of mobility to the red blood cells.

Therefore, the magnetised red blood cell will be able to keep its properties for expression of an antigenic structure while remaining mobile.

Within the framework of the process, markers, and particularly red blood cells, are treated such that their magnetic susceptibility is strongly increased, thus allowing them to migrate in a magnetic field created by a permanent magnet or an electromagnet.

This ‘magnetic marking’ is not done through a magnetised probe molecule but through the use of particles that interact non-specifically with the red blood cell membrane so as to create a large number of low intensity links between the surface of the red blood cell and the magnetic particles.

Consequently, magnetic particles are used that have the characteristics of having a very homogenous size, particularly less than one micron and for example approximately 200 nm, with a high content of ferromagnetic material, for example about 75% by mass and a fairly hydrophobic surface condition.

In one particular example, the markers are larger than the particles that enable the transfer in the magnetic field.

These particles are fixed to the surface of the red blood cell, for example, by means of bovine albumin serum so as to create a large number of non-specific and low intensity links between the surface of the red blood cell and the particles.

The nature of the ‘weak’ interactions between the particles and the markers for which the magnetic susceptibility has to be increased is very much dependent on the surface condition of the particles. This surface condition may be of the hydrophobic and/or hydrophilic type. It is the result of suitable production techniques encouraging one surface condition or another.

Fixation takes place in two steps for this purpose, the first consists of activation of the particles so as to modify their surface condition and the second is to put these activated particles into the presence of a suspension of red blood cells that may or may not have been treated by proteolytic enzymes, so as to create non-specific links between the particles and the red blood cells.

Activation may be done extemporaneously before making contact with the marker, or by fabrication.

The red blood cells thus obtained are attracted by a magnetic field that may thus be used directly, or in one variant they may be treated by enzyme solutions frequently encountered in immuno-hematological tests.

The following describes an embodiment of the process to magnetise red blood cells without damaging the antigens that they carry, in which the particles are activated using a tacking substance comprising a solution of bovine albumin.

Step 1-Activation of Ferromagnetic Particles

Particles type P201 made by the Ademtech company are brought into the presence of an 0.1% solution of bovine albumin (weight by volume) in a PBS buffer with ph 7.2. After incubation for 30 minutes at ambient temperature while stirring (magnetic stirring is to be avoided), particles in suspension are attracted by a magnet and the supernatant in which there are no particles is eliminated. The remaining material consisting of ‘tacked’ particles can be used directly during the red blood cell sensitisation phase.

According to another embodiment, particles may be activated using a wetting agent or a detergent such as cholic acid or Tween 20®), possibly combined with the action of a tacking substance, so as to modify the surface condition of the said particles.

According to another embodiment, this activation may be made by using electro-magnetic radiation such as gamma radiation or UV radiation, that are known to modify plastic type surfaces.

Step 2-Sensitisation of red blood cells

The globular suspension put in a LISS (Low Ionic Strength Solution) with an appropriate concentration (it is possible for example, to work with cellular suspensions with a content of between 0.6 and 10%, prior washed three times or not washed with physiological water), is added to the remaining material consisting of tacked ferromagnetic particles. After perfectly homogenising the suspension (check that there are no more lumps of particles), the suspension is incubated at ambient temperature for thirty minutes while stirring gently and uniformly (the entire reactional volume must be made to move). The red blood cells are then washed with a PBS buffer with ph 7.4 (two washings by centrifuging, three minutes at 500 g). The concentration of the remaining material consisting of sensitised red blood cells may then be modified to suit the analysis made using a LISS buffer.

In one particular example, the ratio between the quantity of particles used and the quantity of red blood cells is between 600 and 1000 to obtain sufficient magnetisation without introducing a risk of degrading antigens present on the surface of the red blood cell. The surface occupied by the particles is typically of the order of 10% of the total area of the red blood cell membrane.

This method provides a means of increasing the magnetic susceptibility of red blood cells without modifying the antigens that they carry.

These red blood cells sensitised by the paramagnetic particles then have the double property of being attracted by a magnetic field and also having blood antigens on their surface (group and phenotype). They can then be used as a reaction support and vector for transport of the antigenantibody pair in an immunological analysis test.

For example, the red blood cells thus obtained may be used in IAR (Irregular Agglutinins Search) type tests, either directly as a reagent or as an analyte, or they may be treated by proteolytic enzymes such as papain, in order to make a so-called enzymatic analysis.

These red blood cells have ferromagnetic particles on their surface that confer a paramagnetic property on them, and can be entrained towards the reactive area of a display device under the action of a magnetic force, so as to enable detection of antibodies directed against antigen determinants present on the surface of the red blood cells.

The red blood cells can also be treated directly using the described process, as an analyte of a blood sample to make them paramagnetic, and thus enable migration of the said red blood cells towards an area capable of detecting the antigens that they support.

In other embodiments, particular elements such as antibodies may be treated so that they can be made paramagnetic using a method similar to the method presented above.

Magnetised antibodies can be used to entrain the said red blood cells, for example to group them.

In other embodiments, chemical markers may be treated using a method similar to the method presented above to make them paramagnetic.

This type of magnetised marker then have the two properties that they can be attracted under the effect of a magnetic field and that they retain a functional surface, to enable coupling of all sorts of chemical or biological molecules.

The process according to the invention enables direct magnetisation of markers, and particularly figured elements, without the use of covalently coupled molecules.

Furthermore, interaction between the particles and the markers is not definitive. Therefore, it is possible that the markers may return to their initial state after desorption of the particles. This desorption step may be done under nonsevere conditions that do not affect the marker surface.

The process also has the advantage of the speed and simplicity of setting up the interaction between markers and particles, simply under the influence of probabilities of encountering elements that should interact.

Furthermore, the particles used are non-biological products with a very long storage duration that is very different from the storage duration of particles functionalised with biological products recognised as being unstable in the long term.

Finally, there is no need to develop a new particle-marker pair for each use, since one type of particles can be used with different markers (red blood cells, blood platelets, other cells, parasites, etc.) with the same surface characteristics (hydrophobic and/or hydrophilic areas).

1. Process for magnetisation of chemical or biological markers using magnetic particles, the said process comprising steps consisting of:
activating magnetic particles so as to modify their surface condition;
putting activated magnetic particles into contact with markers so as to create non-specific links between them.

2. Process according to claim 1, characterised in that magnetic particles are activated using a tacking substance.

3. Process according to claim 2, characterised in that the tacking substance comprises a solution of bovine albumin.

4. Process according to claim 1, characterised in that magnetic particles are activated using a wetting agent or a detergent.

5. Process according to claim 1, characterised in that magnetic particles are activated using an electromagnetic radiation.

6. Process according to any one of claims 1 to 5, characterised in that the markers are cells, for example red blood cells having blood group antigens on their surface.

7. Process according to any one of claims 1 to 5, characterised in that the markers are antibodies.

8. Process according to any one of claims 1 to 7, characterised in that the ratio between the quantity of particles used and the quantity of markers is between 600 and 1000.

9. Process according to any one of claims 1 to 8, characterised in that the size of the particles is less than one micron and for example approximately 200 nm.

10. Use of biological markers magnetised by using a process according to any one of claims 6 to 9 as reagents or analytes in a biological analysis test.
